(19) United States
(12) Patent Application Publication
(10) Pub. No.: US 2012/0184772 A1
(43) Pub. Date: Jul. 19, 2012

(54) ALKYLATION OF TRIIODO-SUBSTITUTED ARYLAMIDES IN AN AQUEOUS MIXED SOLVENT SYSTEM

(76) Inventors: Allan R. Bailey, Ballwin, MO (US); Michelle M. Jones, O'Fallon, MO (US); Tino J. Caviggiola, III, Hazelwood, MO (US); Mills T. Kneller, University City, MO (US); Alexander N. Petrov, St. Peters, MO (US); Michael A. Brown, O'Fallon, MO (US); Benjamin J. Costello, Meath (IE)

(21) Appl. No.: 13/497,318

(22) PCT Filed: Sep. 28, 2010
(86) PCT No.: PCT/US2010/050457

§ 371 Date: Mar. 21, 2012

Related U.S. Application Data
(60) Provisional application No. 61/246,998, filed on Sep. 30, 2009.

(51) Int. Cl. C07C 231/12

(52) U.S. Cl. 564/153

(57) ABSTRACT

The present disclosure is directed to a process for preparing an alkylated triiodo-substituted arylamide, such as iodixanol, the process comprising contacting a triiodo-substituted arylamide, such as 5-acetamido-N,N′-bis(2,3-dihydroxylpropyl)-2,4,6-triiodoisophthalamide, and an alkylating agent in the presence of a base and a mixed solvent system comprising a non-aqueous solvent and water, wherein the volume ratio of the non-aqueous solvent to water is greater than 1:1. The process advantageously enables the concentration of any impurities or undesirable byproduct from the reaction to be reduced, while increasing the yield of the desired reaction product.

### TECHNICAL FIELD

The present disclosure generally relates to an improved process for alkylating a triiodo-substituted arylamide in an aqueous mixed solvent system in which water is the minor component therein. More particularly, the present disclosure is directed to a process for preparing an alkylated triiodo-substituted arylamide, such as iodixanol, the process comprising contacting a triiodo-substituted arylamide, such as 5-acetamido-N,N′-bis(2,3-dihydroxylpropyl)-2,4,6-triiodoisophthalamide, and an alkylating agent in the presence of a base and a mixed solvent system comprising a non-aqueous solvent and water, wherein the volume ratio of the non-aqueous solvent to water is greater than 1:1. The process advantageously enables the concentration of impurities or undesirable byproducts from the reaction to be reduced, while increasing the yield of the desired reaction product.

### BACKGROUND OF THE DISCLOSURE

Diagnostic imaging is an important non-invasive tool for the evaluation of pathology and physiology. More particularly, X-ray imaging is a well known and extremely valuable tool for the early detection and diagnosis of various disease states in the human body. The use of contrast agents and/or media for image enhancement in medical X-ray imaging procedures is widespread. A detailed background on contrast agents and media in medical imaging is provided, for example, by D. P. Swanson et al., Pharmaceuticals in Medical Imaging (1990, MacMillan Publishing Company).

Briefly, in X-ray imaging, transmitted radiation is used to produce a radiograph based upon overall tissue attenuation characteristics. X-rays pass through various tissues and are attenuated by scattering, i.e., reflection or refraction or energy absorption. However, certain body organs, vessels and anatomical sites exhibit so little absorption of X-ray radiation that radiographs of these body portions are difficult to obtain. To overcome this problem, radiologists routinely introduce an X-ray absorbing medium containing a contrast agent into such body organs, vessels and anatomical sites.

The production methods commonly used to prepare iodinated X-ray contrast media or agents typically result in the formation of impurities or byproducts, and/or the presence of unreacted starting components, in the reaction mixture that are difficult to remove. (See, e.g., U.S. Pat. Nos. 5,648,536; 5,204,005; and, 4,396,598; the entire contents of which are incorporated herein by reference for all relevant and consistent purposes.) The presence of these impurities creates a challenge for the manufacturer, at least in part because specifications for such X-ray contrast media or agents impose very low limits on the acceptable amount of such impurities. For example, one impurity that may be encountered in the preparation of iodixanol is the difficult to remove impurity known as “Impurity G”, which has the structure illustrated below.

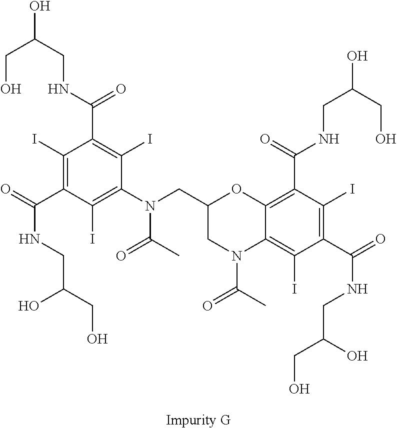

This impurity is formed by cyclization of the hydroxyl group on the alkylating linker, present between the two molecules (or monomers) that formed the dimerized iodixanol, with one of the central aromatic rings, with concomitant loss of iodide.

In the production of contrast media or agents, purification may be achieved by means of crystallization techniques and/or purification columns, in order to remove impurities from the crude product following completion of the synthetic steps (as described in, for example, U.S. Pat. Nos. 4,396,598 and 5,204,005, which teach the preparation and/or purification of triiodo-substituted contrast agents, the entire contents of which is incorporated herein by reference for all relevant and consistent purposes). The cost and time involved in such purification operations, including for example the regeneration and/or replacing of purification column packing, is significant. Large amounts of costly resins and large volumes of solutions are also necessary to regenerate the purification column packing between uses. These costs are significant in the production of various contrast media or agents.

Accordingly, there is a need in the art for a processing method of making iodinated X-ray contrast agents, such as iodixanol, that provides high conversion or yield of the desired product, while reducing the concentration of impurities that are formed in the reaction mixture. This reduction of impurities in the reaction mixture has the added benefit of reducing the costs associated with subsequent isolation or purification of the desired reaction product.

### BRIEF SUMMARY OF THE DISCLOSURE

Briefly, therefore, the present disclosure is generally directed to an improved process for preparing an iodinated X-ray contrast agent, the process comprising alkylating a triiodo-substituted arylamide in an aqueous mixed solvent system in which water is the minor component therein. More particularly, the present disclosure is directed to a process for preparing an iodinated X-ray contrast agent, the process comprising contacting a triiodo-substituted arylamide having the structure of Formula (I):

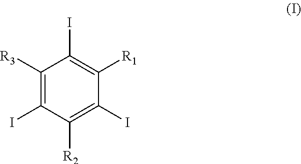

with an alkylating agent in the presence of a base and a mixed solvent system comprising a non-aqueous solvent and water, wherein the volume ratio of the non-aqueous solvent to water is greater than 1:1 and less than about 10:1. In Structure (I), R₁, R₂ and R₃ may be the same or different, and further may be independently selected from —NH—R₅, —C(O)—NH—R₆, or —NH—C(O)—R₆, provided at least one of R₁, R₂ and R₃ has one of the following structures:

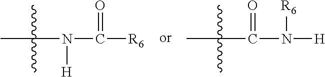

wherein R₅ and R₆ may be the same or different and may be independently selected from hydrogen, or substituted or unsubstituted alkyl, and further provided that R₆ is not hydrogen when R₁, R₂ or R₃ has the former structure (i.e., —NH—C(O)—R₆). In the reaction, the N atom is alkylated to replace the H atom bound thereto with a substituted or unsubstituted alkyl group.

In one particular embodiment, the present disclosure is directed to such a process wherein a dialkylating agent is used. In this or yet another particular embodiment, the present disclosure is directed to a process for preparing the X-ray contrast agent iodixanol, which has the structure of Formula (II).

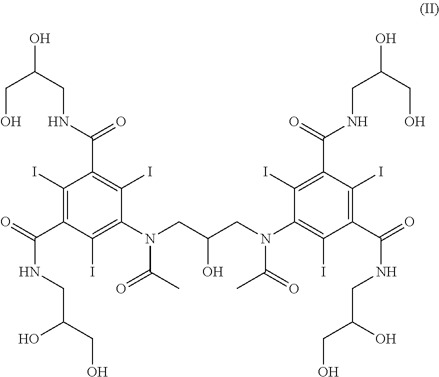

The process comprises contacting the triiodo-substituted arylamide 5-acetamido-N,N′-bis(2,3-dihydroxylpropyl)-2,4,6-triiodoisophthalamide (which may alternatively be referred to herein as “Compound A”), which has the structure of Formula (III),

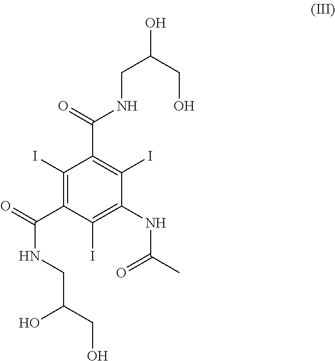

with a dialkylating agent, such as epichlorohydrin, in the presence of a base and a mixed solvent system comprising a non-aqueous solvent and water, wherein the volume ratio of the non-aqueous solvent to water is greater than 1:1.

In a preferred embodiment of one or more of the above processes, the mixed solvent system preferably comprises dimethylacetamide (DMAc) and water, and still more preferably the mixed solvent system comprises these components in a volume ratio of about 2:1, respectively.

### DETAILED DESCRIPTION OF THE DISCLOSURE

As further detailed herein below, it has been discovered that iodinated X-ray contrast agents may be prepared from triiodo-substituted arylamide compounds, such as for example phenylamide compounds, in a reaction process carried out in a mixed solvent system comprising a non-aqueous solvent and water, wherein the non-aqueous solvent is in excess; that is, iodinated X-ray contrast agents may be advantageously alkylated in a mixed solvent system wherein a volume ratio of the non-aqueous solvent to water is greater than 1:1. More particularly, it has been discovered that this mixed solvent system may be used in such a reaction in order to obtain the desired alkylated reaction product in a higher concentration or yield, and/or to decrease the concentration of impurities or undesirable byproducts formed in the reaction (e.g., reduce the concentration of hard to remove impurities or undesirable reaction byproducts in the reaction mixture in which the iodinated X-ray contrast agent or reaction product is formed, thus in turn simplifying the subsequent isolation or purification needed in order to obtain the desired reaction product). In one preferred embodiment, and as further illustrated below, this mixed solvent system may be used to prepare the X-ray contrast agent iodixanol.

### I. X-RAY CONTRAST AGENTS

As previously noted, the present disclosure is generally directed to a process for alkylating triiodo-substituted arylamides having the structure of Formula (I), below, with an alkylating agent in the presence of an appropriate base and a mixed solvent system comprising a non-aqueous solvent and water, wherein the volume ratio of the non-aqueous solvent to water is greater than 1:1 and less than about 10:1.

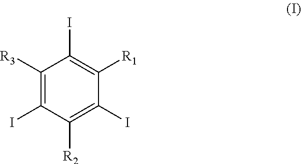

In Structure (I), R₁, R₂ and R₃ may be the same or different, and further may be independently selected from —NH—R₅, —C (O)—NH—R₆, or —NH—C(O)—R₆, provided at least one of R₁, R₂ and R₃ has one of the following structures:

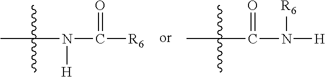

wherein R₅ and R₆ may be the same or different and may be independently selected from hydrogen, or substituted or unsubstituted alkyl, and in various embodiments may be substituted or unsubstituted lower alkyl (e.g., methyl, ethyl, propyl, butyl, pentyl, etc., optionally substituted with one or more heteroatom containing groups, such as for example one or more hydroxyl, alkoxy, amino or amido groups), and further provided that R₆ is not hydrogen when R₁, R₂ or R₃ has the former structure (i.e., —NH—C(O)—R₆). In one preferred embodiment, at least one of R₁, R₂ and R₃ in the triiodo-substituted arylamide of Formula (I) has the structure:

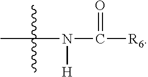

In a more preferred embodiment, however, only one of R₁, R₂ and R₃ has the above-noted structure, the other two having the structure:

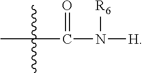

As generally illustrated in Scheme 1 below, for the representative compound of the structure of Formula IV, in the present reaction, a N atom that is part of the amide functionality on the ring is alkylated to replace the H atom bound thereto with a substituted or unsubstituted alkyl group, R₇, derived from the alkylating agent (which is further defined elsewhere herein below), to obtain the compound of the structure of Formula V:

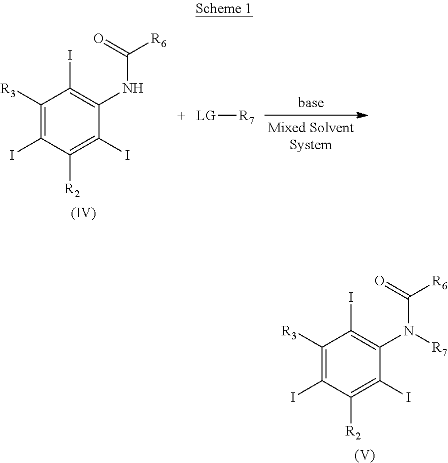

wherein LG is a “leaving group” and R₇ is a substituted or unsubstituted alkyl moiety, as further detailed elsewhere herein.

In this regard it is to be noted that Scheme 1, and the compounds therein, are provide for illustration purposes only, and therefore should not be viewed in a limiting sense. For example, in an alternative embodiment, two or three amide groups may be present on the ring of the structure of Formula IV and, if reacted with multiple molar equivalents of an alkylating agent, one or both may additionally be alkylated in the compound of the structure of Formula (V). In yet another alternative embodiment, a dialkylating agent may be used, with multiple molar equivalents of the starting triiodo-substituted arylamide compound, which may result in the formation of a dimer or dimerized compound (such as in the case of iodixanol, as further illustrated below).

The compounds generally encompassed by the structure of Formula (I) may be obtained commercially, or alternatively they may be prepared using processes and methodologies generally known in the field. For example, in one preferred embodiment of the present disclosure, and as further illustrated by Scheme 2 below, the process may be used to prepare the X-ray contrast agent iodixanol, which has the structure of Formula (II), by reacting the starting compound of the structure of Formula (III), which may alternatively be referred to herein as Compound A, with a suitable dialkylating agent, such as epichlorohydrin, in the presence of a suitable base (e.g., sodium hydroxide) and the mixed solvent system (e.g., DMAc/H₂O).

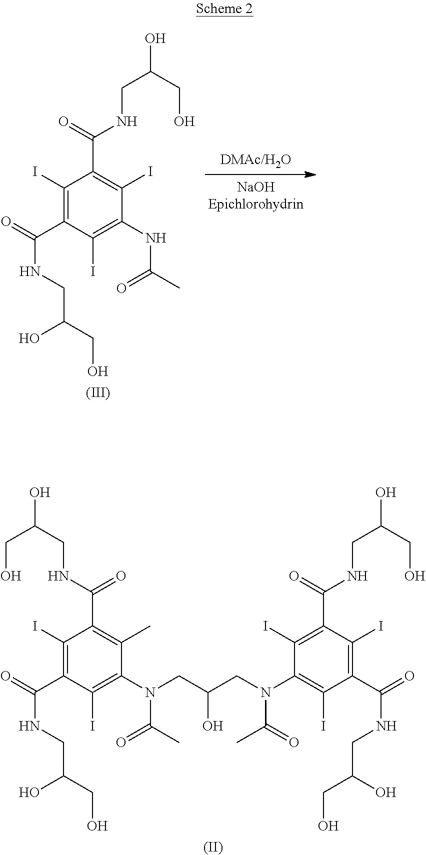

Compound A may be prepared using techniques generally known in the art, such as for example by the process disclosed in U.S. Pat. No. 5,705,692 (the entire contents of which are incorporated herein by reference for all relevant and consistent purposes), and more specifically the process disclosed in Example 1 therein.

### II. MIXED SOLVENT SYSTEM

As noted above, in accordance with the present disclosure, the alkylation of a triiodo-substituted arylamide is carried out in the presence of a mixed solvent system comprising a non-aqueous solvent and water, wherein the non-aqueous solvent is the major component and water is the minor component. In this regard it is to be noted that, as used herein, “mixed solvent system” refers to a solvent system comprising a non-aqueous solvent and water, wherein the concentration of water therein is more than just a trace amount (or is above the level commonly associated with being an impurity). More particularly, however, the mixed solvent system has a volume ratio of a non-aqueous solvent to water that is greater than 1:1 and less than about 10:1, or greater than 1:1 and less than about 5:1, and in various embodiments may be greater than about 1.25:1, about 1.5:1, about 1.75:1, about 2:1, about 2.25:1, or even about 2.5:1, and less than about 10:1, about 5:1, or about 3:1. In one or more preferred embodiments, however, the volume ratio is between 1:1 and about 5:1, or between 1:1 and about 3:1, or between about 1.5:1 and about 2.5:1, or between about 1.75:1 and about 2.25:1, with the volume ratio of about 2:1 being most preferred in one or more embodiments.

Selection of a suitable solvent for use as the non-aqueous solvent component of the mixed solvent system, as well as the ratio of the components in the mixed solvent system, may be made based on such factors as the solubility of the reagents and/or solubility of the resulting reaction products or byproducts (i.e., impurities). For example, solubility of the desired reaction product and undesirable reaction byproducts is a consideration, because differences in solubility in the solvent system may aid with subsequent isolation and/or purification of the desired reaction product. As a result, depending on the reagents, reaction product and/or reaction byproducts, it may be desirable to have a sufficient amount of water present in the mixed solvent system, or the reaction mixture itself, in order to ensure one or more of the reagents, reaction product or reaction byproduct, is dissolved or soluble therein.

In general, however, suitable solvents include those solvents that are at least partially miscible with water, and more particularly are polar organic, or polar aprotic, solvents. Suitable solvents include, for example, dimethylacetamide (DMAc), dimethyl sulfoxide (DMSO), dimethyl formamide (DMF), tetrahydrofuran (THF), acetonitrile (ACN), methanol, 2-methoxyethanol, and isopropanol, as well as some combination thereof. In one or more preferred embodiments, the solvent may additionally be selected in order to obtain a homogeneous or single-phase reaction solution or reaction mixture upon completion of the reaction (as determined, for example, upon expiration of a designated reaction time limit or upon reaching some minimum reaction product concentration in the reaction solution or mixture, as further detailed elsewhere herein). It is generally believed that a single-phase reaction solution enables the reaction product to be more easily isolated, and/or for the undesirable reaction byproducts to be more easily removed. For example, experience to-date has shown that, in the preparation of iodixanol, the combination of DMAc and water advantageously results in a single phase or homogeneous reaction mixture (after the reaction is determined to be completed). In contrast, experience to-date has also shown that, in the preparation of iodixanol, the combination of ACN and water results in a two-phase reaction mixture (after the reaction is determined to be completed). While the presence of a two-phase reaction mixture is not necessarily problematic, it may create the need for additional steps during isolation and/or purification of the reaction product.

Accordingly, in one preferred embodiment of the present disclosure, the solvent system is a mixture of DMAc and water. More preferably, the solvent system comprises, or consists essentially of, DMAc and water, wherein the volume ratio of these two components is between 1:1 and about 5:1, or between about 1.5:1 and about 2.5:1, or between about 1.75:1 and about 2:25:1, with a ratio of about 2:1 being most preferred.

### III. ALKYLATING AGENTS AND BASES

As previously noted, the X-ray contrast agents of the present disclosure are produced by an alkylation reaction, wherein a triiodo-substituted arylamide is contacted with an alkylation agent in the presence of a mixed solvent system and a base. A number of alkylating agents are generally known in the art, and selection from among these for use in the process of the present disclosure may be made based on such consideration as, for example: (i) sufficient reactivity with the amide functionality, and more particularly the nitrogen atom of the amide functionality, of the triiodo-substituted arylamide compound, such that alkylation may occur; (ii) appropriate composition of the alkyl group, which is transferred from the alkylating agent to the triiodo-substituted arylamide compound; and/or (iii) sufficient solubility in the mixed solvent system.

Typically, however, the alkylating agent (e.g., LG-R₇), or dialkylating agent (e.g., LG-R₇-LG), will be selected from the group consisting of those agents that comprise between 1 and about 10 carbon atoms, or between 1 and about 6 carbon atoms, or between 1 and about 5 carbon atoms, and may be in the form of an open chain or ring (e.g., cycloalkyl or heterocycloalkyl). One or more of the carbon atoms of the alkylating agent may be substituted with one or more heteroatoms (e.g., substituents selected from, for example, halo, hydroxyl and alkoxy), which serves as a “leaving group” (LG), in the reaction and is thus displaced from the alkyl group or moiety that is transferred from the alkylating agent to the nitrogen atom of the triiodo-substituted arylamide. In various embodiments, the agent may in particular be selected from those agents comprising an alkyl chain (or alternatively a cycloalkyl or heterocycloalkyl ring) substituted with one or two halogen atoms (e.g., fluoro, chloro, bromo, etc.), and/or one or two hydroxyl groups, and/or one or two alkoxy groups (e.g., methoxy, ethoxy, etc.), the alkyl chain, or alternatively cycloalkyl or heterocycloalkyl ring, typically comprising from between about 1 and about 5, or about 2 and about 4, carbon atoms therein.

Accordingly, in various embodiments the agent may be either a mono-alkylating agent, or a dialkylating agent (the agent for example having two reactive sites and thus enabling two molecules of a triiodo-substituted arylamide to be linked together). In a particularly preferred embodiment, the alkylating agent may selected from the group consisting of monohalo- or dihalo-substituted alkanols or dialkanols (e.g., 1,3-dihalo-2-propanol, such as 1,3-dichloro-2-propanol, or 1-halo-2,3-propane diol, such as 1-chloro-2,3-propane diol), any of which may optionally be further substituted with an alkoxy group, such as a methoxy group (e.g., 1-halo-3-alkoxy-propanol, such as 1-chloro-3-methoxy-2-propanol), as well as various halo-substituted heterocycloalkyl compounds (e.g., epichlorohydrin or glycidol).

In addition to the alkylating (or dialkylating) agent and the starting triiodo-substituted arylamide, as well as the mixed solvent system, the reaction mixture additionally comprises a base. Generally speaking, essentially any base may be used that will enable the alkylating reaction to be carried out in a satisfactory way (e.g., sufficient reaction product yield, and/or purity). Typically, however, the base will be selected from known metal hydroxides (e.g., sodium hydroxide, potassium hydroxide, etc.), metal carbonates (e.g., sodium carbonate, potassium carbonate, etc.), and strong organic bases (i.e., bases which act to raise the pH of the reaction mixture to about 10 or more, as detailed elsewhere herein below).

As previously noted, the molar ratio of the starting compound (i.e., the compound of Formula (I)), the alkylating or dialkylating agent, and/or base, may be determined or optimized using means generally known in the art, in order to maximize purity and/or yield of the desired product. Typically, however, the molar ratio of starting compound to alkylating agent will be between about 1:3 (e.g., when multiple sites on the triiodo-substituted compound are to be alkylated) and about 2:1 (e.g., when two molecules of the starting compound are reacted with a single molecule of a dialkylating agent, in order to form a dimer), with ranges of about 1:2 to about 2:1, or about 1:1 to about 2:1, or about 1.5:1 to about 2:1, being more commonly employed. In one preferred embodiment, wherein about 2 molar equivalents of the starting compound are to be reacted with or linked by means of about 1 molar equivalent of a dialkylating agent, a slight molar excess of the dialkylating agent may be used, to for example offset the slight consumption of dialkylating agent by the base. Accordingly, in such an embodiment the molar ratio of the starting compound to the dialkylating agent may be about 2:1.1, about 2:1.15, or about 2:1.2.

With respect to the molar ratio of the starting compound to base, it is to be noted that the present reaction may be viewed as being somewhat self-catalyzing. As a result, one equivalent of base per equivalent of starting compound, or site of alkylation when multiple sites on the starting compound may be alkylated, may not be needed. Conversely, the use of too much base may quench the alkylating agent and/or increase the concentration of impurities or the number of side reactions that occur in the reaction mixture. As a result, in one or more embodiments of the present disclosure, the molar ratio of starting compound to base may be about 1:0.5, about 1:0.6, about 1:0.8, or even about 1:1, the molar ratio for example being in the range of about 1:0.5 to 1:1, or about 1:0.6 to 1:1. In one or more alternative embodiments, however, the molar ratio of the starting compound to base may be between about 1:1 (e.g., when only one site on the triiodo-substituted compound is to be alkylated) and less than about 1:3 (e.g., when multiple sites on the triiodo-substituted compound are to be alkylated), or between about 1:1 and about 1:2.5.

### IV. REACTION CONDITIONS AND PROCESS STEP

The process of the present disclosure generally involves forming a reaction mixture comprising the mixed solvent system, the base, the alkylating (or dialkylating) agent, and the triiodo-substituted arylamide starting compound. In the process, the order of addition of the components is not narrowly critical; that is, the base, the alkylating agent and triiodo-substituted compound may be added to the solvent system in essentially any order. Preferably, however, the reaction mixture is formed by initially mixing or slurrying together the triiodo-substituted arylamide compound, the base and the mixed solvent system. After agitating this mixture or slurry for a given period of time (e.g., at least about 30 minutes, 60 minutes or even 90 minutes), the alkylating agent is added. The pH of the reaction mixture may optionally be adjusted before or after addition of the alkylating agent as needed, in order to maximize or optimize the reaction (e.g., to increase reaction product yield and/or limit the formation of impurities). In one or more embodiments, the pH of the reaction mixture may be monitored and adjusted before or during the reaction, to ensure the pH is within the range of about 10 and about 14, or about 11 and about 13 (as determined using means known in the art).

Once the reaction mixture is formed, the reaction mixture may be heated or cooled as needed to maintain the reaction mixture within a desired temperature range for a desired period of time. For example, in one embodiment, the temperature of the reaction mixture will be maintained within the range of from about 0° C. to about 75° C., or from about 5° C. to about 60° C., or from about 10° C. to about 50° C., or from about 20° C. to about 40° C.

The reaction time, or more specifically the time the reaction mixture is maintained within the desired temperature range, may be set based on a number of factors, such as the concentration of the desired reaction product in the reaction mixture or the concentration of unwanted impurities or byproducts in the reaction mixture (as determined using means generally known in the art, including for example withdrawing an aliquot of the reaction mixture and subjecting it to a known analytical method, such as HPLC, to measure the concentration of the desired reaction product or unwanted impurity or byproduct therein). Typically, however, the reaction time will be between about 5 hours and about 75 hours, or between about 10 hours and about 50 hours, or between about 15 hours and about 25 hours.

In this regard it is to be noted that the order of addition, the reaction temperature, reaction mixture pH, and/or the reaction time or duration, may be other than herein described without departing from the scope of the present disclosure.

### V. REACTION PRODUCT ISOLATION AND YIELD

Once the reaction has reached the desired end point (as determined, for example, by passage of a sufficient amount of time or by means of analytical analysis), the reaction may be stopped or quenched using means generally known in the art. For example, in one particular embodiment, the reaction may be stopped or quenched by the addition of an appropriate amount of an acid (e.g., a hydrochloric acid). Additionally, means generally known in the art may be used to take the reaction mixture forward, in order to isolate and purify the desired reaction product as needed. For example, in one particular embodiment, the reaction mixture is processed using means generally known in the art (e.g., distillation, solvent separation or extraction, etc.) to remove the non-aqueous component of the mixed solvent system (e.g., the DMAc). The remaining, essentially aqueous, solution may then be further processed by adding additional water (in order, for example, to ensure all components therein are thoroughly dissolved), followed by subjecting the solution to de-salting and deionizing techniques generally known in the art, prior to final purification of the reaction product.

Advantageous, by the proper selection of components of the mixed solvent system and the relative ratios therebetween, the process of the present disclosure enables the desired reaction product to be obtained in a yield of about 40%, about 50%, about 60%, about 70%, or more, based on the total weight of the reaction product mixture (i.e., the mixture obtained upon completion of the reaction to form the reaction product), the yield for example being within the range of about 40% to about 70% or about 50% to about 60%. The process of the present disclosure additionally enables the desired reaction product (e.g., iodixanol) to be obtained, after the reaction product has been isolated and purified by means generally known in the art, having an overall impurity concentration of less than about 5 area %, about 4 area %, about 3 area %, about 2 area % or even about 1 area % (relative to the reaction product itself, as determined by means generally known in the art, including for example high performance liquid chromatography techniques). Stated another way, the reaction product (e.g., iodixanol), after isolation and purification by means generally known in the art, may be obtained having a purity of at least about 95 area %, about 96 area %, about 97 area %, about 98 area %, about 99 area %, or more.

Additionally, or alternatively, the process of the present disclosure advantageously (i) enables the concentration of one or more undesirable reaction impurities or byproducts (e.g., difficult to remove impurities, such as one or more starting compounds or over-alkylated reaction byproducts, as well as, in the case of iodixanol, Impurity G and/or iohexyl) in the reaction product mixture to be reduced by limiting their formation, and/or (ii) simplifies subsequent purification of the reaction product (by, for example, eliminating or reduced the concentration of hard to remove impurities in the reaction product mixture, such as those previously noted). For example, by proper selection of the mixed solvent system, removal of impurities, such as unreacted starting components (such as the triiodo-substituted arylamide, or Compound A in the case of iodixanol), and/or reaction byproducts or salts (e.g., iohexyl, when the desired reaction product is iodixanol), and/or hard to remove impurities (e.g., over-alkylated compounds, and/or Impurity G), may be simplified, by for example preventing or limiting their formation, and/or ensuring that such impurities (such as, in the case of iodixanol, starting Compound A, or over-alkylated compounds, or iohexyl) remain in solution, with or without the reaction product (i.e., the reaction product may or may not remain in solution).

Accordingly, in one or more embodiments, the present process yields a reaction product mixture, prior to any isolation or purification of the reaction product therein, that has an overall or total concentration of impurities of about 60 wt %, about 50 wt %, about 40 wt %, about 30 wt % or less, wherein “impurity” as used herein generally refers to any detectable compound that is not the desired reaction product, and in particular is unreacted starting components, reaction byproducts, and/or salts thereof, present in the reaction product mixture. More particularly, the present process may yield a reaction product mixture that contains: (i) less than about 50 wt %, about 40 wt %, about 30 wt %, about 20 wt %, about 10 wt %, or even about 5 wt % of a starting reagent or component (e.g., Compound A in the case of iodixanol); and/or (ii) less than about 15 wt %, about 10 wt %, or about 5 wt % of an over-alkylated reaction byproduct (e.g., an reaction byproduct wherein more sites on the triido-substituted arylamide are alkylated than desired); and/or (iii) less than about 15 wt %, about 10 wt %, or about 5 wt % of an under-alkylated or not fully reacted compound or reaction byproduct (e.g., iohexyl, in the case of iodixanol); and/or (iv) less than about 5 wt %, about 2.5 wt %, or about 1 wt % of a bicyclic impurity (e.g., Impurity G, in the case of iodixanol).

In this regard it is to be noted that the reaction yield, and/or purity (or impurity concentration), as well as the concentration and type of impurities present in the reaction product mixture, may be other than herein described without departing from the scope of the intended invention.

### VI. DEFINITIONS

The compounds described herein may have asymmetric centers. Compounds of the present disclosure containing an asymmetrically substituted atom may be isolated in optically active or racemic form. All chiral, diastereomeric, racemic forms and all geometric isomeric forms of a structure are intended, unless the specific stereochemistry or isomeric form is specifically indicated. All processes used to prepare compounds of the present disclosure and intermediates made therein are considered to be part of the present disclosure.

As used herein, “optional” or “optionally” means that the subsequently described event or circumstance may or may not occur, and that the description includes instances where the event or circumstance occurs and instances where it does not.

The term “amido” as used herein includes substituted amido moieties where the substituents include, but are not limited to, one or more of aryl and C₁₋₂₀ alkyl, each of which may be optionally substituted by one or more aryl, carbaldehyde, keto, carboxyl, cyano, halo, nitro, C₁₋₂₀ alkyl, phosphorous-oxo acid, sulfur-oxy acid, hydroxyl, oxy, mercapto, and thio substituents.

The term “amino” as used herein includes substituted amino moieties where the substituents include, but are not limited to, one or more of aryl and C₁₋₂₀ alkyl, each of which may be optionally substituted by one or more aryl, carbaldehyde, keto, carboxyl, cyano, halo, nitro, C₁₋₂₀ alkyl, phosphorous-oxo acid, sulfur-oxy acid, hydroxyl, oxy, mercapto, and thio substituents.

The terms “aryl” or “ar” as used herein, alone or as part of another group, denote optionally substituted homocyclic aromatic groups, preferably monocyclic or bicyclic groups containing from 6 to 12 carbons in the ring portion, such as phenyl, biphenyl, naphthyl, substituted phenyl, substituted biphenyl or substituted naphthyl. Phenyl and substituted phenyl are the more preferred aryl.

The term “arylamide” as used herein refers to aromatic compounds having one or more amide or amido substituents thereon. Phenyl and substituted phenyl are the more preferred amide or amido substituted rings.

The terms “halogen” or “halo” as used herein alone or as part of another group refer to chlorine, bromine, fluorine, and iodine.

Unless otherwise indicated, the “alkyl” groups described herein are preferably lower alkyl containing from one to 10 carbon atoms in the principal chain, and up to 20 carbon atoms. They may be straight or branched chain or cyclic (e.g., cycloalkyl) and include methyl, ethyl, propyl, isopropyl, butyl, pentyl, hexyl and the like. Accordingly, the phrase “C₁₋₂₀ alkyl” generally refers to alkyl groups having between about 1 and about 20 carbon atoms, and includes such ranges as about 1 to about 15 carbon atoms, about 1 to about 10 carbon atoms, or about 1 to about 5 carbon atoms.

The term “substituted” as in “substituted arylamide” or “substituted alkyl” and the like, means that in the group in question (i.e., the amine, the alkyl, or other moiety that follows the term), at least one hydrogen atom bound to a nitrogen atom or carbon atom, respectively, is replaced with one or more substituent groups such as hydroxy, alkoxy, alkylthio, phosphino, amino, halo, silyl, and the like. When the term “substituted” introduces a list of possible substituted groups, it is intended that the term apply to every member of that group. That is, the phrase “substituted alkyl, alkenyl and alkynyl” is to be interpreted as “substituted alkyl, substituted alkenyl and substituted alkynyl.” Similarly, “optionally substituted alkyl, alkenyl and alkynyl” is to be interpreted as “optionally substituted alkyl, optionally substituted alkenyl and optionally substituted alkynyl.”

The term “alkanol” refers to an alkyl group having a hydroxy group or substituent thereon. The term “dialkanol” refers to an alkyl group having two hydroxy groups or substituents therein.

The modifiers “hetero” and “heteroatom-containing”, as in “heteroalkyl” or “heteroatom-containing group” refer to a molecule or molecular fragment in which one or more carbon atoms in the main or primary chain, or backbone, is replaced with a heteroatom. Thus, for example, the term “heteroalkyl” refers to an alkyl group that contains a heteroatom in the main or primary chain, while “heterocycloalkyl” reference to a cycloalkyl group that contains a heteroatom in the backbone of the ring. When the term “heteroatom-containing” introduces a list of possible heteroatom-containing groups, it is intended that the term apply to every member of that group.

### EXAMPLES

The following examples are provided to further illustrate the present disclosure, and therefore should not be viewed in a limiting sense. In this regard it is to be noted, in particular, that while orders of addition are not necessarily critical, the reactions in these examples were carried out as follows: A reaction vessel or flask was charged with, in order, DMAc (or other co-solvent, where applicable), Compound A, water, sodium hydroxide, and stirred until Compound A dissolved (or until the amount dissolved was stable). If applicable, HCl was then charged, followed by epichlorohydrin. Stirring was continued at room temperature (unless otherwise noted) for the desired amount of time.

### Examples 1-8

### Preparation of Iodixanol—Various DMAc/H₂O Ratios

As further detailed in Table 1, below, in this series of Examples (1-8, which includes Comparative Examples 5-8, wherein the ratio of non-aqueous solvent to water is equal to or less than 1:1), iodixanol was prepared by coupling two moles of Compound A (i.e., 5-acetamido-N,N′-bis(2,3-dihydroxypropyl)-2,4,6-triiodoisophthalamide) with epichlorohydrin in a solvent mixture of water and DMAc. For purposes of comparing the impact of the mixed solvent system on the reaction, in each example only the concentration of the components of the mixed solvent system (i.e., the concentration of water or DMAc in the solvent mixture) was changed. The reaction time (approximately 19 hours), temperature, reagent concentrations and ratios were maintained relatively constant (the amount of reagents charged being adjusted based on the w/w assay of the Compound A: 0.72 eq. NaOH, 0.34 eq. epichlorohydrin).

TABLE 1

| | | | | | | | Over- | |
|---|---|---|---|---|---|---|---|---|
| | H₂O | DMAc | Acetylated | Compd | | | alkylated | Imp. |
| Exp. | Vol. % | Vol. % | Iohexol | A | Iohexol | Iodixanol | Imp. | G |
| 1 | 10 | 90 | 5.14 | 49.79 | 1.07 | 36.79 | 0.30 | 2.38 |
| 2 | 22 | 78 | 2.39 | 44.95 | 1.66 | 44.87 | 0.78 | 1.38 |
| 3 | 34 | 66 | 0.48 | 44.20 | 2.60 | 46.84 | 0.97 | 0.76 |
| 4 | 34 | 66 | — | 43.13 | 2.62 | 49.23 | 1.01 | 0.95 |
| 5 | 50 | 50 | 0.43 | 43.58 | 4.04 | 46.83 | 1.54 | 0.36 |
| 6 | 66 | 34 | — | 45.35 | 5.10 | 44.95 | 1.62 | 0.35 |
| 7 | 78 | 22 | — | 46.04 | 5.75 | 43.73 | 1.76 | 0.32 |
| 8 | 90 | 10 | — | 46.86 | 6.10 | 42.99 | 1.56 | 0.13 |

These results indicate the highest conversion to iodixanol is obtained from a 2:1 volume ratio of DMAc:H₂O. Also, the results show that increasing the concentration of H₂O in the mixed solvent system increases the amount of iohexyl in the reaction mixture, and reduces the amount of Impurity G that is formed. Advantageously, when the reaction was carried out in the presence of excess Compound A, in order to increase the effective yield and decrease impurities formed or present therein, Compound A still present therein remained in solution after the reaction was complete.

### Comparative Examples 9-10

### Compound A Coupling—High Conversion (H₂O)

As further detailed in Table 2, below, in these Comparative Examples (9-10), iodixanol was prepared by coupling two moles of Compound A (i.e., 5-acetamido-N,N′-bis(2,3-dihydroxypropyl)-2,4,6-triiodoisophthalamide) with epichlorohydrin in a solvent of water only. The composition of the reaction mixture was measured during the reaction (22.5 hours into the reaction) and at the time the reaction was stopped (46 hours). As compared to previous Examples 1-8, these reactions were carried out using comparably more epichlorohydrin and less water in the reaction mixture (i.e., reagent concentration in the reaction mixture was higher), in order to achieve a higher conversion (i.e., more Compound A converted to iodixanol).

TABLE 2

| | | | Over- | | | | Over- | |
|---|---|---|---|---|---|---|---|---|
| | NaOH | Epi. | alkylated | Compd | | | alkylated | |
| Exp. | (eq.) | (eq.) | Iodixanol | A | Iohexol | Iodixanol | Imp. | Imp. G |
| 9 | 0.65 | 0.31 | | 61.73 | 3.39 | 16.60 | 1.18 | 0.45 |
| 22.5 hr. | | 0.31 | 0.18 | 18.21 | 13.67 | 57.18 | 4.20 | 0.35 |
| | | (5 hr.) | | | | | | |
| 46 hr. | — | — | 0.41 | 12.51 | 17.53 | 60.56 | 5.39 | 0.31 |
| 10 | 0.65* | 0.31 | | 56.61 | 3.95 | 32.02 | 1.23 | 0.33 |
| 22.5 hr. | | 0.31 | 0.20 | 9.71 | 13.90 | 66.10 | 4.34 | 0.30 |
| | | (5 hr.) | | | | | | |
| 46 hr. | — | — | 0.50 | 5.60 | 16.34 | 68.22 | 5.77 | 0.19 |

*The reaction was back titrated with HCl.

It is to be noted that while the actual reaction time was longer here, as compared to the reaction times in Examples 1-8 (carried out for only 19 hours), the effective reaction times are roughly the same, due to the higher charge of epichlorohydrin that was used. Accordingly, the results here, when compared to those from Examples 1-8, suggest reaction rate is more dependent on reaction temperature and/or total payload or concentration.

It is to be further noted that the concentration of iohexyl is higher in these reactions, when comparing the relative amounts here versus Examples 1-8. However, the iodixanol yield here is also higher, and carrying out the reaction under “high conversion” conditions helps to eliminate the need to recover unreacted Compound A.

### Examples 11-14

### Compound A Coupling—High Conversion (DMAc/H₂O)

As further detailed in Tables 3A and 3B, below, in these Examples (11-14), iodixanol was prepared by coupling two moles of Compound A (i.e., 5-acetamido-N,N′-bis(2,3-dihydroxypropyl)-2,4,6-triiodoisophthal-amide) with epichlorohydrin in a mixed solvent system of DMAc and water (2:1 ratio DMAc to water). The composition of the reaction mixture was measured multiple times during the reaction, as well as at the time the reaction was stopped.

TABLE 3A

| | | | Over- | | | | Over- | |
|---|---|---|---|---|---|---|---|---|
| | NaOH | Epi. | alkylated | Compd | | | alkylated | |
| Exp. | (eq.) | (eq.) | Iodixanol | A | Iohexol | Iodixanol | Imp. | Imp. G |
| 11 | 0.65 | 0.31 | — | 53.85 | 2.04 | 35.57 | — | — |
| 22.5 hr. | | 0.31 | — | 10.39 | 7.94 | 68.72 | 2.82 | 1.17 |
| | | (5 hr.) | | | | | | |
| 48 hr. | — | — | 0.11 | 3.97 | 11.55 | 72.88 | 4.27 | 2.34 |
| 119 hr. | — | — | 0.11 | 2.48 | 13.71 | 72.28 | 4.54 | 3.06 |
| 119 hr. | — | — | — | 2.56 | 13.72 | 71.83 | 4.20 | 2.79 |
| 12 | 0.65* | 0.31 | — | 53.31 | 4.21 | 35.24 | — | — |
| 22.5 hr. | — | 0.31 | 0.10 | 8.65 | 10.34 | 69.44 | 2.94 | 1.50 |
| | | (5 hr.) | | | | | | |
| 48 hr. | — | — | 0.24 | 4.34 | 12.43 | 70.92 | 4.19 | 3.22 |
| 118 hr. | — | — | 0.23 | 3.41 | 12.98 | 69.46 | 5.35 | 4.58 |

*The reaction was back titrated with HCl.

TABLE 3B

| | | | | | | | Over- | |
|---|---|---|---|---|---|---|---|---|
| | NaOH | Epi. | Solvent | Comp | | | alkylated | |
| Exp. | (eq.) | (eq.) | (mL/g) | A | Iohexol | Iodixanol | Imp. | Imp. G |
| 13 | 0.67 | 0.32 | 2 | 63.33 | 1.33 | 25.99 | 0.16 | 0.21 |
| 23.5 hr. | — | 0.32 | — | 7.90 | 8.69 | 69.76 | 2.99 | 1.40 |
| | | (5 hr.) | | | | | | |
| 48 hr. | — | — | — | 2.95 | 12.17 | 72.20 | 4.33 | 2.77 |
| 14 | 0.69 | 0.33 | 2 | 62.07 | 2.82 | 26.21 | 0.72 | 0.32 |
| 23.5 hr. | — | 0.33 | — | 6.32 | 9.75 | 69.46 | 4.33 | 1.96 |
| | | (5 hr.) | | | | | | |
| 48 hr. | — | — | — | 2.29 | 12.03 | 70.02 | 5.56 | 3.73 |

It is to be noted that, here epichlorohydrin was added in two portions (as compared, for example, to a single addition as in Examples 9-10). As a result, the effective reaction time here was comparably longer (because the concentration of epichlorohydrin in the reaction mixture was lower). The results illustrate that the addition of epichlorohydrin in this way, or maintaining a lower epichlorohydrin concentration in the reaction mixture (as compared to Examples 9-10) increases the iodixanol yield and decreases the level or concentration of impurities.

Additionally, it is to be noted that the reactions seemed to reach a plateau before about 120 hours (see, e.g., the 48 hour results, in terms of iodixanol %, are higher than the 118 or 119 hour results).

### Examples 15.16

### Compound A Coupling—Low Conversion (DMAc/H₂O)

As further detailed in Table 4, below, in these Examples (15-16), iodixanol was prepared by coupling two moles of Compound A (i.e., 5-acetamido-N,N′-bis(2,3-dihydroxypropyl)-2,4,6-triiodoisophthalamide) with epichlorohydrin in a mixed solvent system of DMAc and water (2:1 ratio). In these Examples, the reaction was stopped much sooner than the previous examples detailed above.

TABLE 4

| | | | | | | | Over- | |
|---|---|---|---|---|---|---|---|---|
| | NaOH | Epi. | Solvent | Comp | | | alkylated | |
| Exp. | (eq.) | (eq.) | (mL/g) | A | Iohexol | Iodixanol | Imp. | Imp. G |
| 15 | 0.72 | 0.34 | 3 | — | — | — | — | — |
| 19 hr. | — | — | — | 41.99 | 3.48 | 49.81 | 1.11 | 1.35 |
| filtrate | — | — | — | 22.54 | 5.14 | 64.96 | 2.05 | 2.33 |
| 16 | 0.72 | 0.34 | 3 | — | — | — | — | — |
| 19 hr. | — | — | — | 42.08 | 3.60 | 49.20 | 1.02 | 1.33 |
| filtrate | — | — | — | 20.10 | 5.17 | 68.75 | 2.19 | 1.90 |
| solid | — | 20.55 | — | — | — | — | — | — |

These reactions were stopped early in order to examine how the concentration of impurities could be minimized. Here, it was observed that while iodixanol yields were slightly lower (as compared to earlier Examples), the concentration of impurities (Impurity G and iohexyl) was significantly lower.

### Examples 17-19

### Compound A Coupling—High Conversion (ACN/H₂O)

As further detailed in Tables 5A and 5B, below, in these Examples (17-19), iodixanol was prepared by coupling two moles of Compound A (i.e., 5-acetamido-N,N′-bis(2,3-dihydroxypropyl)-2,4,6-triiodoisophthal-amide) with epichlorohydrin in a mixed solvent system of ACN and water (2:1 ratio).

TABLE 5A

| | | | | | | | Over- | |
|---|---|---|---|---|---|---|---|---|
| | NaOH | Epi. | Solvent | Compd | | | alkylated | |
| Exp. | (eq.) | (eq.) | (mL/g) | A | Iohexol | Iodixanol | Imp. | Imp. G |
| 17 | 0.65 | 0.31 | 3 | — | — | — | — | — |
| 74.5 hr. | — | 0.31 | — | 12.44 | 11.25 | 62.62 | 7.89 | 1.56 |
| | | (5 hr.) | | | | | | |
| 18 | 0.65* | 0.31 | 3 | — | — | — | — | — |
| 22.5 hr. | — | 0.31 | — | 61.98 | 2.46 | 31.84 | 1.23 | 0.32 |
| 46.5 hr. | — | 0.31 | — | 27.23 | 6.73 | 58.17 | 3.49 | 0.67 |
| 70.5 hr. | — | — | — | 8.45 | 11.01 | 68.06 | 5.95 | 1.04 |

*The reaction was back titrated with HCl.

TABLE 5B

| | | | | | | | Over- | |
|---|---|---|---|---|---|---|---|---|
| | NaOH | Epi. | Solvent | Compd | | | alkylated | |
| Exp. | (eq.) | (eq.) | (mL/g) | A | Iohexol | Iodixanol | Imp. | Imp. G |
| 19 | 0.72* | 0.34 | 3 | — | — | — | — | — |
| 5 hr. | — | 0.34 | — | 83.82 | 0.70 | 11.82 | 0.71 | 0.19 |
| 23 hr. | — | 0.29 | — | 29.41 | 5.45 | 57.57 | 2.73 | 0.49 |
| 45 hr. | — | — | — | 6.79 | 10.57 | 65.06 | 8.33 | 1.55 |

*The reaction was back titrated with HCl.

The results illustrate that the ACN/water system resulted in a higher conversion and lower levels of Impurity G than the DMAc/water system. Additionally, however, the ACN/water mixed solvent system did create some challenges in terms of product isolation (the ACN/water system is biphasic and heterogeneous until late in the reaction).

### Comparative Example 20

### Compound A Coupling in Water

As further detailed in Table 6, below, in this Example iodixanol was prepared by dissolving approximately 2 equivalents of Compound A (i.e., 5-acetamido-N,N′-bis(2,3-dihydroxypropyl)-2,4,6-thiodoisophthalamide sodium salt) and 1.23 equivalents of sodium hydroxide in water (4 ml/g of compound A). Hydrochloric acid (0.43 eq) was added to adjust the pH to approximately 12, and 0.38 equivalents of epichlorohydrin was charged thereto. The reaction was carried out at room temperature (approximately 23° C.).

TABLE 6

| Experiment | Compd A | Iohexol | Iodixanol | Other |
|---|---|---|---|---|
| 20 | 18.2 | 12.9 | 55.7 | 0.1 |

The results illustrate that when no DMAc is added, iodixanol yields can be lower by approximately 10-20%. Additionally, while the absolute amount of impurities in the mixture was comparable to DMAc/water, relative to the yield of iodixanol it was considerably higher.

### Examples 21

### Compound A Coupling—In DMAc/Water

As further detailed in Table 7, below, in this Example iodixanol was prepared by mixing 15.0 grams of Compound A (i.e., 5-acetamido-N,N′-bis(2,3-dihydroxypropyl)-2,4,6-triiodoisophthalamide) with 0.33 equivalents of epichlorohydrin (0.523 mL) in a mixed solvent of water (10 mL) and DMAc (20 mL), along with 1.23 equivalents of sodium hydroxide (1.30 mL) and 0.53 equivalents of HCl solution (0.874 mL).

TABLE 7

| Rxn | | Cmpd | | | Overalkylated | |
|---|---|---|---|---|---|---|
| Time | Intermediate | A | Iohexol | Iodixanol | Imp. | Imp. G |
| 20.5 hr. | 0.07 | 39.40 | 4.33 | 52.04 | 1.38 | 0.64 |

It is to be noted that, in comparison to previous Examples, slightly different results are obtained when using excess sodium hydroxide and adjusting pH with HCl (as compared, for example, to when a smaller charge of sodium hydroxide is used alone, such as in Examples 15-16).

Although the present disclosure has been described with reference to preferred embodiments, persons skilled in the art will recognize that changes may be made in form and detail without departing from the spirit and scope of the disclosure.

When introducing elements of the present disclosure or the embodiments(s) thereof, the articles “a”, “an”, “the” and “said” are intended to mean that there are one or more of the elements. The terms “comprising”, “including” and “having” are intended to be inclusive and mean that there may be additional elements other than the listed elements.

What is claimed is:

1. A process for preparing an iodinated X-ray contrast agent, the process comprising contacting a triiodo-substituted arylamide having the structure of Formula (I): 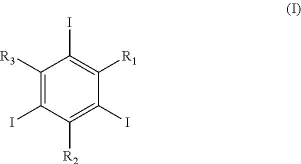
with an alkylating agent in the presence of a base and a mixed solvent system comprising a non-aqueous solvent and water, wherein the volume ratio of the non-aqueous solvent to water is greater than 1:1 and less than about 10:1, and further wherein:
(i) R₁, R₂ and R₃ may be the same or different, and may be independently selected from —NH—R₅, —C(O)—NH—R₆, or —NH—C(O)—R₆, provided at least one of R₁, R₂ and R₃ has one of the following structures: 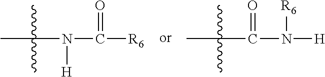
(ii) R₅ and R₆ may be the same or different and may be independently selected from hydrogen, or substituted or unsubstituted alkyl, and further provided that R₆ is not hydrogen when R₁, R₂ or R₃ has the structure —NH—C(O)—R₆; and,
(iii) in the reaction, the N atom is alkylated to replace the H atom bound thereto with a substituted or unsubstituted alkyl group from the alkylating agent.

2. The process as set forth in claim 1, wherein at least one of R₁, R₂ and R₃ in the triiodo-substituted arylamide of Formula (I) has the structure:

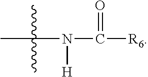

3. The process as set forth in claim 2, wherein only one of R₁, R₂ and R₃ has the structure, 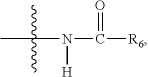
while the other two have the structure:

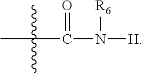

4. The process as set forth in claim 1, wherein the volume ratio of non-aqueous solvent to water is between greater than 1:1 and less than about 5:1.

5. The process as set forth in claim 1, wherein the volume ratio of non-aqueous solvent to water is about 2:1.

6. The process as set forth in one of the preceding claims, wherein the non-aqueous solvent is a polar, aprotic solvent.

7. The process as set forth in claim 6, wherein the polar, aprotic solvent is selected from the group consisting of N,N-dimethylacetamide, dimethyl sulfoxide, dimethyl formamide, and combinations thereof.

8. The process as set forth in one of the preceding claims, wherein the non-aqueous solvent is N,N-dimethylacetamide.

9. The process as set forth in one of the preceding claims, wherein the alkylating agent has a formula LG-R₇, wherein LG is a leaving group selected from the group consisting of halogen, hydroxyl, and alkoxyl, and R₇ is an alkyl group.

10. The process as set forth in one of the preceding claims, wherein the alkylating agent is a dialkylating agent having a formula LG-R₇-LG, wherein each LG is a leaving group independently selected from the group consisting of halogen, hydroxyl, and alkoxyl, and R₇ is an alkyl group.

11. The process as set forth in one of the preceding claim 9 or 10, wherein the alkylating agent is selected from the group consisting of 1,3-dichloro-2-propanol, 1-chloro-2,3-propane diol, 1-chloro-3-methoxy-2-propanol, and epichlorohydrin.

12. The process as set forth in one of the preceding claims, wherein the base is selected from the group consisting of sodium hydroxide, potassium hydroxide, sodium carbonate, or potassium carbonate.

13. The process as set forth in one of the preceding claims, wherein the molar ratio of the triiodo-substituted arylamide compound and the alkylating compound is between about 2:1 and about 1:3.

14. The process as set forth in claim 13, wherein the molar ratio of the triiodo-substituted arylamide compound and the alkylating compound is between about 2:1 and about 2:1.2.

15. The process as set forth in claim 14, wherein the molar ratio of the triiodo-substituted arylamide compound and the alkylating compound is about 2:1.15.

16. The process as set forth in one of the preceding claims, wherein the molar ratio of the triiodo-substituted arylamide compound and the base is between about 1:0.5 and about 1:1.

17. The process as set forth in one of preceding claims 1 to 15, wherein the molar ratio of the triiodo-substituted arylamide compound and the base is between about 1:1 and about 1:3.

18. The process as set forth in one of the preceding claims, wherein the amide compound is 5-acetamido-N,N′-bis(2,3-dihydroxypropyl)-2,4,6-triiodoisophthalamide.

19. The process as set forth in one of the preceding claims, wherein the iodinated X-ray contrast media reaction product is iodixanol.

20. The process as set forth in one of the preceding claims, wherein a reaction mixture is formed of the triiodo-substituted arylamide, the alkylating agent, the base and the mixed solvent system is maintained at a temperature between about 10° C. to about 50° C.
